(19) United States
(12) Patent Application Publication
(10) Pub. No.: US 2018/0296423 A1
(43) Pub. Date: Oct. 18, 2018

(54) JOINT-ASSISTING DEVICE

(71) Applicant: WEARATEK CO., LTD., Daejeon (KR)

(72) Inventor: Chong Kun PARK, Siheung-si, Gyeonggi-do (KR)

(21) Appl. No.: 15/574,043

(22) PCT Filed: May 18, 2016
(86) PCT No.: PCT/KR2016/005259

§ 371 Date: Nov. 14, 2017

(30) Foreign Application Priority Data
May 22, 2015 (KR) 10-2015-0071589

(51) Int. Cl. A61H 3/00; A61F 5/01

(52) U.S. Cl. CPC A61H 3/00; A61F 5/0102; A61H 2201/165; A61H 2201/1253; A61H 2201/164; A61H 2201/1614; A61F 2005/0179; A61H 2201/1673

(57) ABSTRACT

The present invention provides a joint-assisting device comprising: a wearing part worn on the waist of a body; connecting parts provided at both sides of the lower part of the wearing part so as to be fixed around the hip joints; rotating parts worn on the body at the upper side of the wearing part so as to rotate while moving integrally with the body during bending or stretching motions of the upper body or knees, coupled to the connecting parts by rotary shafts provided at one side thereof, having rotary pieces formed in a fan shape so as to rotate around the rotary shafts, and having guide grooves formed along the arcs of the rotary pieces; and an elastic part of which the upper part is coupled to the connecting part so as to reduce the weight applied to the lumbar or joints when the rotating parts rotate, and which comprises an elastic member embedded in a state in which the upper end or lower end thereof is fixed, a connecting cable connecting the elastic member and the rotary piece and moving in a state, in which the connecting cable is settled in the guide groove, when the rotating part rotates, and elastic fixing bands provided at the lower part of the elastic part so as to be worn on the thighs.

### TECHNICAL FIELD

The present invention relates to a joint assisting device. More particularly, the present invention relates to a joint assisting device that is worn on the body to reduce a weight applied to the lumbar or joints through the expansion or compression of an elastic member when the upper body or knee are bent or stretched.

### BACKGROUND ART

Since a human body gently connects bones forming a skeleton via joints, various movements of the human body may be performed by bending or stretching around the joints. Particularly, the arms, legs an waist of the human body are representative body regions at which the joints are bent or stretched. In recent years, there are many patients who suffer from various diseases due to the inadequate joint movement of these body regions.

For example, when a standing person bends his or her upper body to raise a heavy object or bends his or her knees, the load of the human body concentrates on the lumbar or knees, so that a weight is applied to the lumbar or joints. As such, if the weight is continuously or repeatedly applied to the lumbar or joints of the human body, the cartilage of the knee or the like is worn or damaged, thus causing arthritis. Thus, when a person walks or moves, he or she feels considerable pain or finds it uncomfortable to walk.

As the related art, Korean Patent Publication No. 10-1154434 (Jun. 15, 2012) has disclosed an angle adjusting device for a knee joint-assisting device, in which a fixing pin coupled to a bending-angle operating pin is simultaneously coupled to a fixing groove formed in a bending-angle adjusting disc and a fixing groove formed in a stretching-angle adjusting disc, thus regulating a movement.

However, the related art is problematic in that it has a complicated structure, so that it is difficult to manufacture and a manufacturing cost is high, and force is directly applied to the joints to cause exercise effects, so that the joints may be strained, and separate manipulation is required when in use, so that it is complicated to use, and various body sizes and motions of wearers are not considered, so that it is inconvenient to wear.

### DISCLOSURE

### Technical Problem

An object of the present invention is to provide a joint assisting device, which has a relatively simple structure, thus guaranteeing easy manufacture as well as convenient handling and use, and reducing a manufacturing cost, and which supplements energy consumed in the lumbar or joints movements in a state where force is not directly applied to the joints, thus reducing a weight applied to the joints, and which does not require separate manipulation, thus enabling convenient use, and which is suitable for various body sizes of wearers and is applicable to individual motions of left and right sides of the body.

### Technical Solution

In order to accomplish the above object, the present invention provides a joint-assisting device including a wearing part worn on the waist of a body; a connecting part provided at each of both sides of a lower part of the wearing part to be fixed around the hip joint; a rotating part worn on the body at an upper side of the wearing part to rotate while moving integrally with the body during bending or stretching motions of the upper body or knees, coupled to the connecting part by a rotary shaft provided at one side thereof, having a rotary piece formed in a fan shape to rotate around the rotary shaft, and having a guide groove formed along an arc of the rotary piece; and an elastic part coupled at an upper part thereof to the connecting part to reduce a weight applied to the lumbar or joints when the rotating part rotates, and including an elastic member embedded in a state in which an upper or lower end thereof is fixed, a connecting cable connecting the elastic member and the rotary piece and moving in a state in which the connecting cable is seated in the guide groove, when the rotating part rotates, and an elastic fixing band provided at a lower part of the elastic part to be worn on the thigh.

Further, the present invention provides a joint-assisting device including a pair of wearing parts worn on the legs of a body; a connecting part provided at a left or right side of an upper part of each of the wearing part to be fixed around the knee joint; a rotating part worn on the body at a lower side of the wearing part to rotate while moving integrally with the body during bending or stretching motions of the knees, coupled to the connecting part by a rotary shaft provided at one side thereof, having a rotary piece formed in a fan shape to rotate around the rotary shaft, and having a guide groove formed along an arc of the rotary piece; and an elastic part coupled at a lower part thereof to the connecting part to reduce a weight applied to the knee joint when the rotating part rotates, and including an elastic member embedded in a state in which an upper or lower end thereof is fixed, a connecting cable connecting the elastic member and the rotary piece and moving in a state in which the connecting cable is seated in the guide groove, when the rotating part rotates, and an elastic fixing band provided at an upper part of the elastic part to be worn on the thigh.

### Advantageous Effects

According to the present invention, a joint assisting device has a relatively simple structure to guarantee easy manufacture as well as convenient handling and use and to reduce a manufacturing cost, and supplements energy consumed in the lumbar or joints movements by expanding or compressing an elastic member in a state where force is not directly applied to the joints, thus reducing a weight applied to the joints, and allows an angle at which left and right sides are connected to be adjusted by a fastening member when a wearer wears the joint assisting device, thus being suitable for various body sizes of wearers and applicable to individual motions of left and right sides of the body, and adjusts the tension of the elastic member via a tension adjusting member, thus being efficiently used according to the height or weight of a wearer.

### BRIEF DESCRIPTION OF THE DRAWINGS

The accompanying drawings are intended to illustrate the preferred embodiments of the invention, and aid in understanding the technical spirit of the invention when reading the following description. It is to be interpreted that the invention is not limited to embodiments shown in the drawings.

FIG. 1 is a perspective view illustrating a joint assisting device according to an embodiment of the present invention;

FIGS. 2A, 2B and 2C are a side view, a front view and a rear view illustrating a state in which the joint assisting device according to the embodiment of the present invention is worn, respectively;

FIGS. 3A and 3B are views illustrating an elastic member of the joint assisting device according to the embodiment of the present invention, in which the elastic member is a tension spring in FIG. 3A and is a compression spring in FIG. 3B;

FIGS. 4A, 4B and 4C are a side view, a front view and a rear view illustrating a state in which the joint assisting device according to the embodiment of the present invention is worn, respectively;

FIGS. 5A, 5B and 5C are a side view, a front view and a rear view illustrating a state in which the joint assisting device according to the embodiment of the present invention is worn, respectively;

FIGS. 6A, 6B and 6C are a side view, a front view and a rear view illustrating a state in which the joint assisting device according to the embodiment of the present invention is worn, respectively;

FIGS. 7A and 7B are views illustrating an elastic member of the joint assisting device according to the embodiment of the present invention, in which the elastic member is a tension spring in FIG. 7A and is a compression spring in FIG. 7B;

FIGS. 8A, 8B and 8C are a side view, a front view and a rear view illustrating a state in which a joint assisting device according to another embodiment of the present invention is worn, respectively;

FIGS. 9 and 10 are views illustrating an operation of the joint assisting device according to another embodiment of the present invention; and

FIGS. 11A, 11B and 11C are a side view, a front view and a rear view illustrating a state in which a joint assisting device according to a further embodiment of the present invention is worn, respectively.

### BEST MODE FOR THE INVENTION

Exemplary embodiments of the present invention will be described below in more detail with reference to the accompanying drawings.

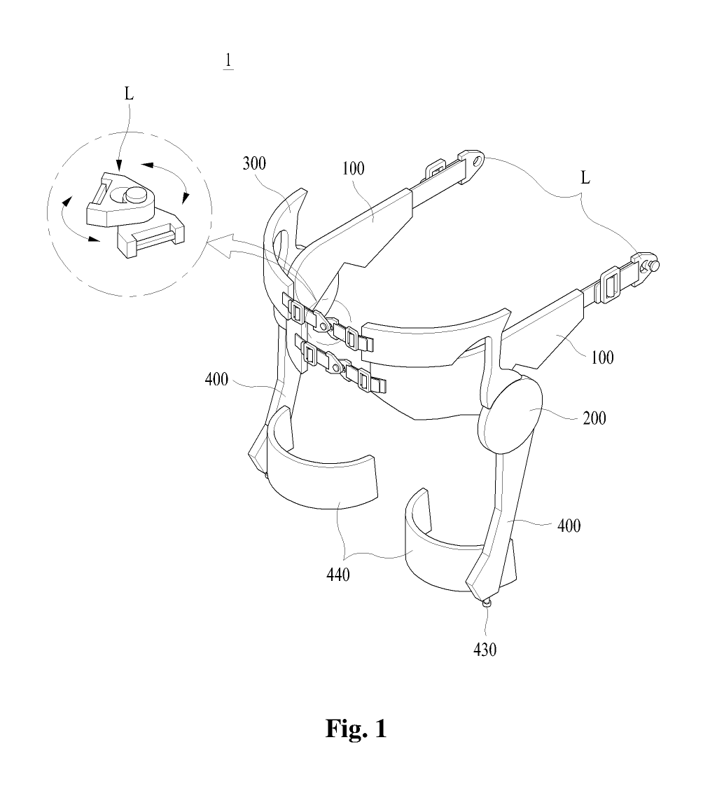

As illustrated in FIGS. 1 to 3, the present invention is directed to a joint assisting device 1 that is worn on a person's body to reduce a weight applied to the lumbar or joints by expanding or compressing an elastic member during the bending or stretching motions of the upper body or knees. The joint assisting device includes a wearing part 100, a connecting part 200, a rotating part 300, and an elastic part 400.

The wearing part 100 is worn on the waist of a wearer body, has a shape similar to that of a general waist belt, and is fastened to allow opposite ends thereof to be separated from each other.

Here, the fastening member L is provided to connect the opposite ends of the wearing part 100 to each other. The fastening member L is rotatably provided on a center of the wearing part 100 to adjust an angle at which both sides of the wearing part 100 are connected.

In addition, the wearing part 100 has a length adjusting member to adjust the length of the wearing part 100 according to a wearer's waist size and thereby allow the device to be easily worn.

Thus, the device is fit for various body sizes of wearers, and is applied to individual motions for left and right sides of a wearer's body by rotating the fastening member L and thereby adjusting an angle at which both sides of the wearing part 100 are fastened, thus minimizing interference and obviating inconvenience.

The connecting parts 200 are provided on both sides of a lower part of the wearing part 100 to be fixed around left and right hip joints of a wearer. Preferably, each connecting part is in the form of a pivot joint. More preferably, the size of each connecting part is reduced to minimize interference in a wearer's body motion or movement.

The rotating part 300 is fixedly coupled with each connecting part 200 via a rotary shaft 310 to be rotatable in a state where the rotating part is coupled to the connecting part 200. A rotary piece 320 has a fan shape and is formed on a side of the rotating part 300 to rotate around the rotary shaft 310.

In this regard, the rotary piece 320 has a fan shape, and a guide groove H is formed along an arc of the rotary piece 320 to guide the upward or downward movement of a connecting cable 420, which will be described later, when the rotating part 300 rotates in a state where the connecting cable 420 is seated in the guide groove H.

Further, the other side of the rotating part 300 is worn on a wearer's body at an upper position of the wearing part 100. That is, the other side of the rotating part 300 is worn on a wearer's body, so that the other side of the rotating part 300 moves integrally with his or her body as if the other side of the rotating part 300 were fixedly coupled to his or her body.

Thus, during the bending or stretching motions of a wearer's upper body or knees, the other side of the rotating part 300 moves integrally with the wearer's body, so that the rotary piece 320 formed on one side rotates upwards or downwards around the rotary shaft 310.

The fastening member L is provided to connect ends of the pair of the rotating parts 300 to each other. The fastening member L is rotatably provided between the rotating parts 300, thus adjusting an angle at which both sides of the pair of rotating parts 300 located at left and right sides of a wearer's body are connected.

In addition, the rotating part 300 has a length adjusting member to adjust the length of the rotating part 300 according to a wearer's waist size and thereby allow the device to be easily worn.

Thus, the device is fit for various body sizes of wearers, and is applied to individual motions for left and right sides of a wearer's body by rotating the fastening member L and thereby adjusting an angle at which both sides of the rotating parts 300 are fastened, thus minimizing interference and obviating inconvenience.

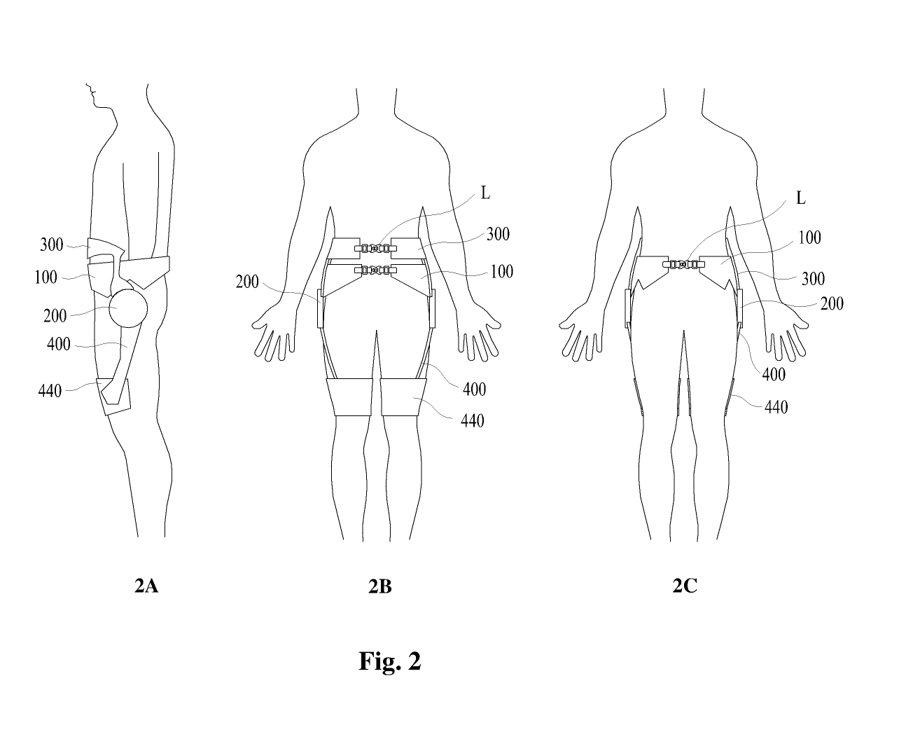

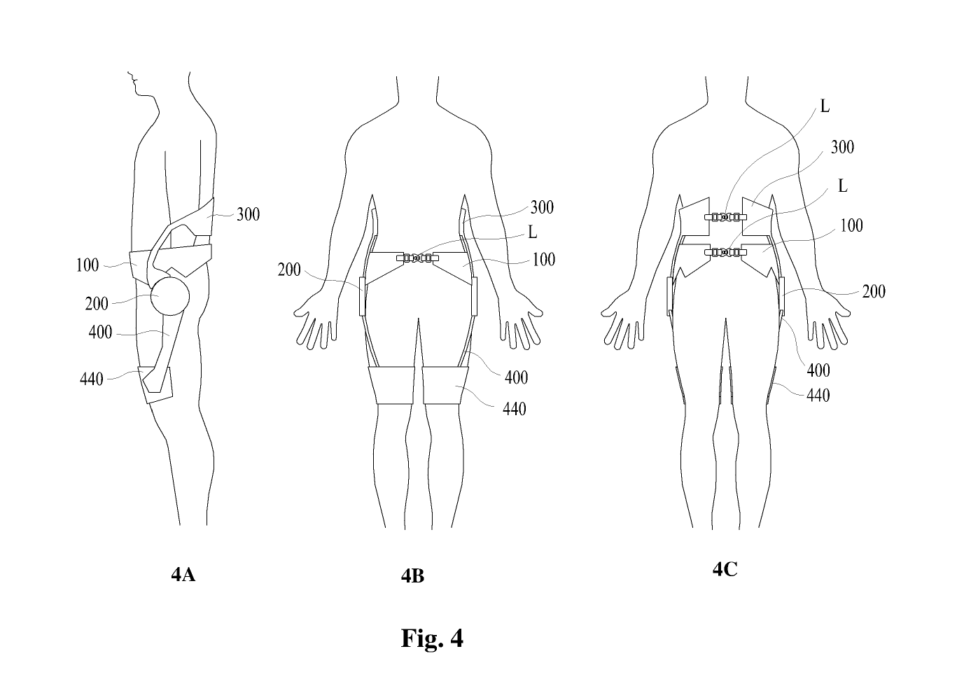

As illustrated in FIGS. 2 to 4, the other side of the rotating part 300 is worn on a wearer's body at the upper position of the wearing part 100. The other side of the rotating part 300 extends through a wearer's flank to the abdominal region or the back so that the device may be worn on his or her abdomen or back.

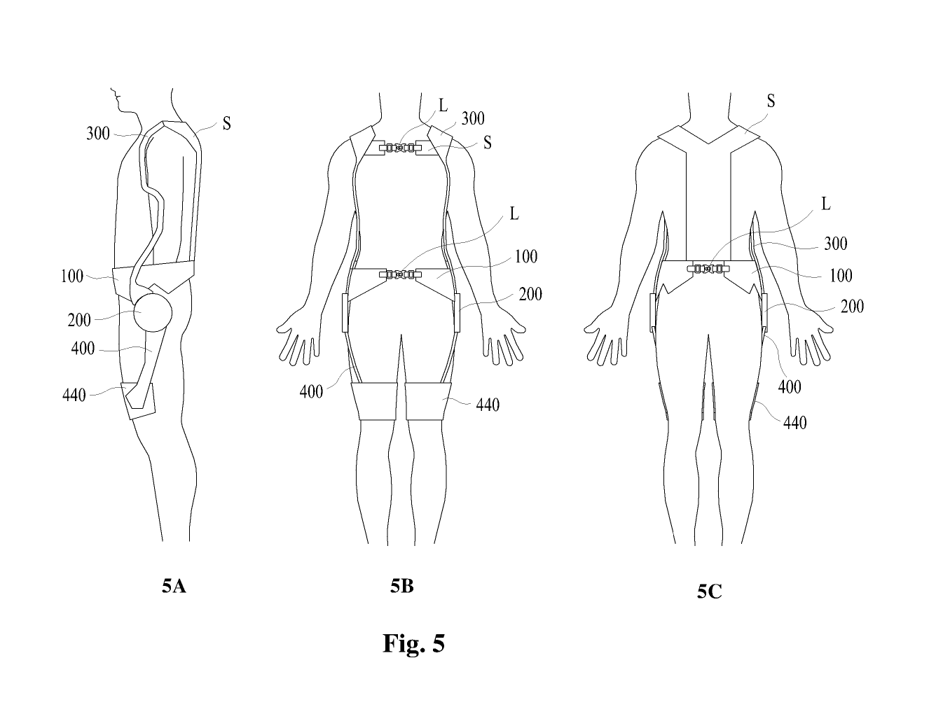

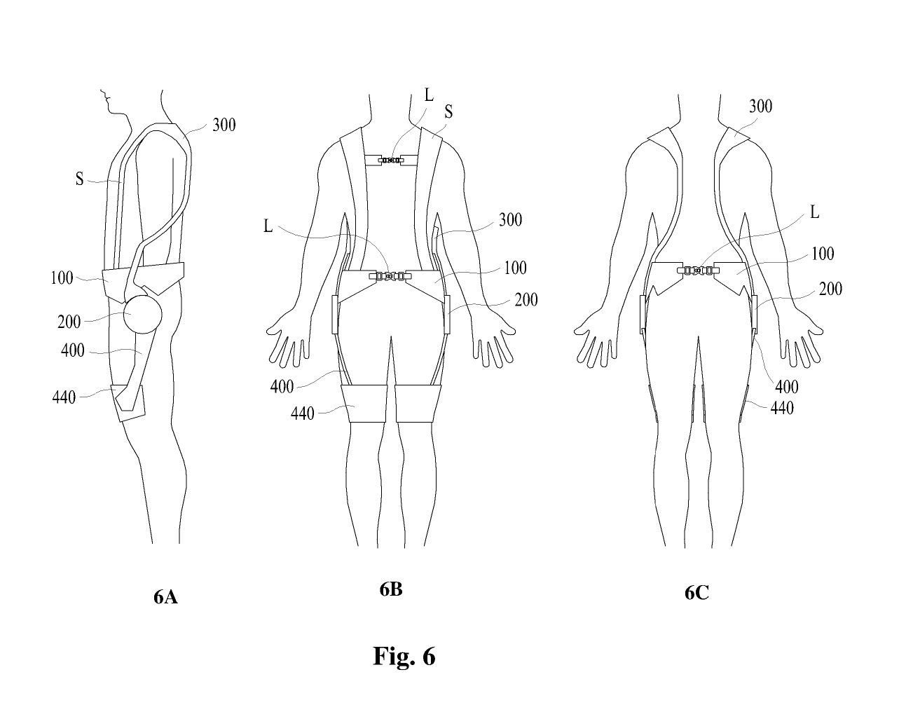

As illustrated in FIGS. 5 and 6, the joint assisting device 1 further includes a shoulder wearing part S that is coupled at one side thereof to the wearing part 100 and held at the other side on the shoulder. The rotating part 300 is worn at the other side on a wearer's body at the upper position of the wearing part 100. The other side of the rotating part 300 extends through a wearer's flank along the chest or the back to the shoulder and then is coupled to the other side of the shoulder wearing part S to be worn on the wearer's body.

Here, the fastening member L is provided to connect both ends of the shoulder wearing part S to each other. The fastening member L is rotatably provided in a center of the shoulder wearing part S to adjust an angle at which both sides of the shoulder wearing part S are connected.

In addition, the shoulder wearing part S has a length adjusting member to adjust the length of the shoulder wearing part S according to a wearer's body size and thereby allow the device to be easily worn.

Thus, the device is fit for various body sizes of wearers, and is applied to individual motions for left and right sides of a wearer's body by rotating the fastening member L and thereby adjusting an angle at which both sides of the shoulder wearing part S are fastened, thus minimizing interference and obviating inconvenience.

As such, the rotating part 300 may be worn on various body regions of a wearer, so that the joint assisting device 1 is selectively worn on a desired body region according to a kind of motion, an environment under which a wearer moves, or a motion habit. Thereby, the rotation of the rotating part 300 integrally worn on a wearer' body leads to the expansion and compression of the elastic member 410 that will be described later, thus supplementing energy consumed in the lumbar or joints movements due to the wearer's motion or movement and thereby reducing a weight applied to the joints.

In addition, in consideration of a body region to be exercised, the rotating part 300 is selectively applied to various body regions of a wearer and then it is possible to exercise a desired body region by rotating the rotating part 300 and thereby expanding or compressing the elastic member 410 that will be described later.

The elastic part 400 is connected to the rotating part 300 coupled to the connecting part 200 to generate energy through expansion and compression when the rotating part 300 rotates and thereby reduce a weight applied to the lumbar or joints when a wearer's upper body or knees are bent or stretched, and includes an elastic member 410, a connecting cable 420 and a tension adjusting member 530.

The elastic member 410 comprises a tension spring or a compression spring, and is embedded in the elastic part 400 in a state where an upper end or a lower end thereof is fixed to the inside of the elastic part 400. Here, when the elastic member 410 comprises the tension spring, its lower end is preferably fixed to the inside of the elastic part 400. When the elastic member comprises the compression spring, its upper end is preferably fixed to the inside of the elastic part 400.

The connecting cable 420 is fixed to the upper end of the elastic member 410 and the rotary piece 320 to connect the rotary piece 320 with the elastic member 410 and thereby operate them together. When the rotating part 300 rotates, the connecting cable moves along the arc of the rotary piece 320. Here, the movement of the connecting cable 420 is guided in a state where the connecting cable is seated in the guide groove H, so that the connecting cable moves upwards or downwards during the rotation of the rotating part 300.

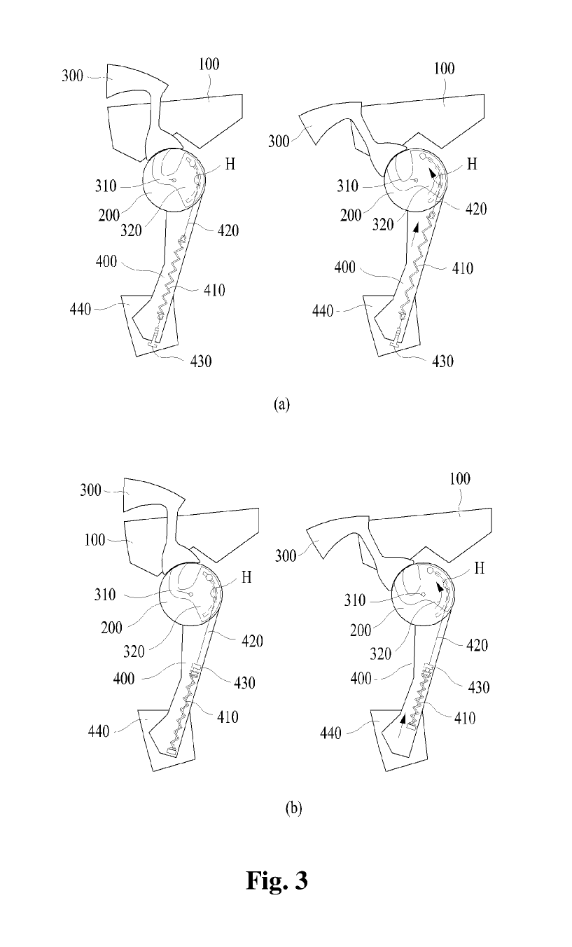

For example, as illustrated in FIG. 3A, if the elastic member 410 is the tension spring, when a wearer bends his or her upper body or knees, the rotary piece 320 rotates upwards by a predetermined angle and the connecting cable 420 moves upwards. Then, the elastic member 410 is expanded upwards in a state where its lower end is fixed to the inside of the elastic part 400, so that energy is accumulated in the elastic member 410.

If a wearer stretches out his or her upper body or knees in this state, the rotary piece 320 rotates downwards by a predetermined angle contrary to the case where the upper body or knees are bent, so that the connecting cable 420 moves downwards, and simultaneously a restoring force is generated in a state where the lower end of the elastic member 410 is fixed to the inside of the elastic part 400, and thereby downward compression is performed. Thus, energy accumulated in the elastic member 410 is released and then is transmitted to a wearer's body. This supplements energy consumed in the lumbar or joints movements, thus reducing a weight applied to the joints.

Meanwhile, as illustrated in FIG. 3B, if the elastic member 410 is the compression spring, when a wearer bends his or her upper body or knees, the rotary piece 320 rotates upwards by a predetermined angle and the connecting cable 420 moves upwards. Then, the elastic member 410 is expanded upwards in a state where its upper end is fixed to the inside of the elastic part 400, so that energy is accumulated in the elastic member 410.

If a wearer stretches out his or her upper body or knees in this state, the rotary piece 320 rotates downwards by a predetermined angle contrary to the case where the upper body or knees are bent, so that the connecting cable 420 moves downwards, and simultaneously a restoring force is generated in a state where the upper end of the elastic member 410 is fixed to the inside of the elastic part 400, and thereby downward compression is performed. Thus, energy accumulated in the elastic member 410 is released and then is transmitted to a wearer's body. This supplements energy consumed in the lumbar or joints movements, thus reducing a weight applied to the joints.

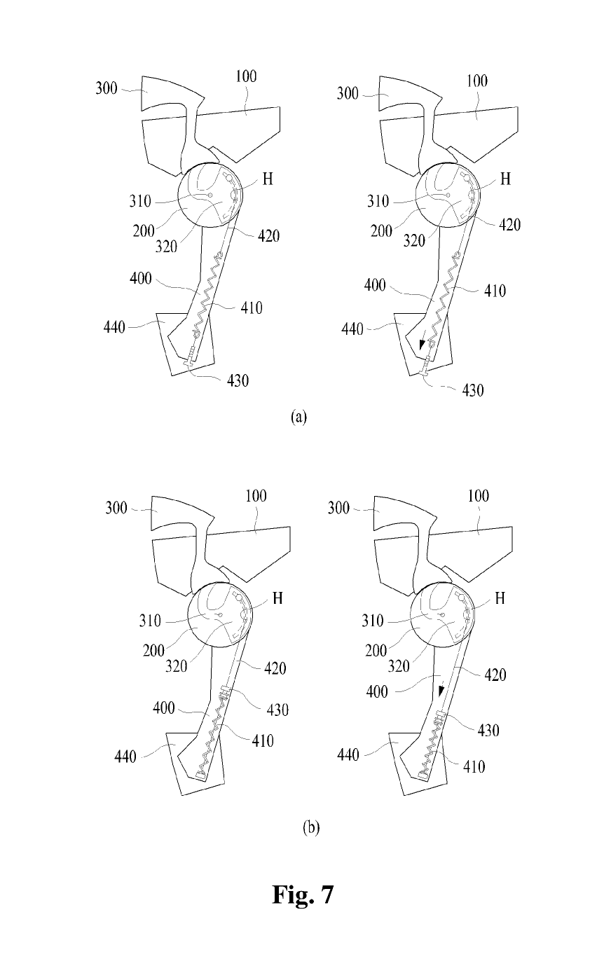

As illustrated in FIG. 7, the tension adjusting member 430 is connected to the upper or lower end of the elastic member 410 so that the upper or lower end of the elastic member 410 is fixed to the tension adjusting member 430. Thus, the elastic member 410 is embedded in the elastic part 400 in a state where the upper or lower end of the elastic member is fixed to the tension adjusting member 430.

In such a state, the length of the elastic member 410 that is the tension spring or the compression spring is adjusted by pushing the tension adjusting member 430 from above or pulling the tension adjusting member downwards, thus adjusting the tension of the elastic member 410 generated by the rotation of the rotating part 300.

Here, as illustrated in FIG. 7A, when the elastic member 410 is the tension spring, the tension adjusting member 430 comprises a rotating bolt connected with the lower end of the elastic member 410 in a state where it communicates with a lower end of the elastic part 400. Thus, it is preferable to adjust the length of the elastic member 410 by pulling the lower end of the elastic member 410 inside the elastic part 400. When the rotary piece 320 rotates upwards, the upper end of the elastic member 410 expands upwards in a state where the lower end thereof is fixed to the tension adjusting member 430.

Further, as illustrated in FIG. 7B, when the elastic member 410 is the compression spring, the tension adjusting member 430 has a double bolt structure to push the upper end of the elastic member 410 in a state where the tension adjusting member is interposed in the elastic part 400. Thus, it is preferable to adjust the length of the elastic member 410 by pushing the upper end of the elastic member 410. When the rotary piece 320 rotates upwards in a state where the upper end of the elastic member 410 is fixed to the tension adjusting member 430 and the connecting cable 420 passes through the elastic member 410 and then is fixed to a washer on a lower side of the elastic member 410, the connecting cable 420 moves upwards and a lower part of the elastic member 410 is drawn through the washer on the lower side of the elastic member 410, so that the lower end of the elastic member 410 is compressed upwards.

In addition, the elastic part 400 further includes an elastic fixing band 440. The upper part of the elastic part is fixedly coupled to the connecting part 200 while the lower part thereof is fixedly coupled to the elastic fixing band. The elastic fixing band 440 is worn on a front of a wearer's thigh. Thus, when the wearer bends or stretches the upper body or knees in a state where the lower part of the elastic part 400 is fixed via the elastic fixing band 440 to the front of the wearer's thigh, the elastic fixing band 440 absorbs a restoring force generated in the elastic member 410, so that the position of the elastic part 400 is firmly held and the joint assisting device 1 is stably used.

A joint assisting device 1 according to another embodiment of the present invention includes the wearing part 100, the connecting part 200 and the rotating part 300, as in the preceding embodiment.

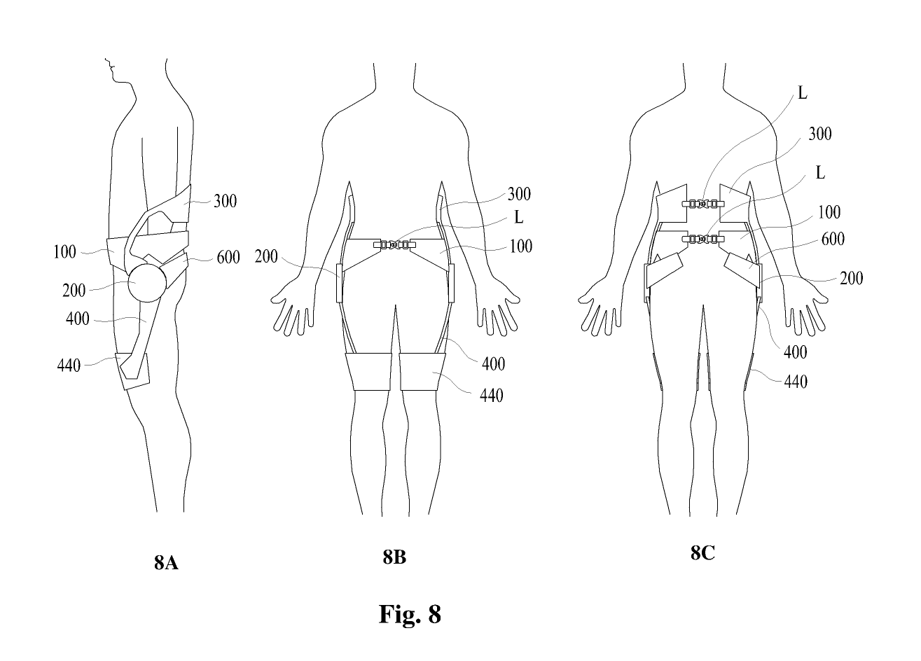

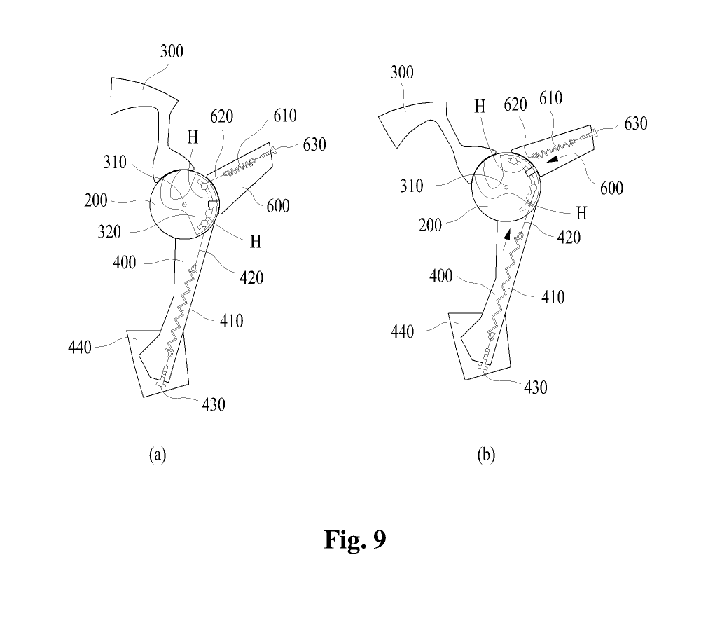

However, as illustrated in FIGS. 8 and 9, this device includes an elastic part 400 and a second elastic part 600 that are connected to the connecting part 200 and the rotating part 300, respectively.

The second elastic part 600 is similar in configuration and function to the elastic part 400. However, the second elastic part is fixed at one end thereof to a lower side of the wearing part 100, unlike the elastic part 400 that is fixedly coupled at a lower part thereof to the elastic fixing band 440.

That is, the joint assisting device 1 according to another embodiment of the present invention is configured to include the elastic part 400 as well as the second elastic part 600, thus supplementing energy consumed in the lumbar or joints movements due to the wearer's motion or movement, thereby reducing a weight applied to the joints or allowing a desired body region of a wearer to be more efficiently exercised.

Here, each of connecting cables 420 and 620 of the elastic part 500 and the second elastic part 600 is fixed to the rotary piece 320, is connected to the rotary piece 320 and the respective elastic members 410 and 610 to be operated together, and moves along the arc of the rotary piece 320 when the rotating part 300 rotates. It is preferable that the connecting cables are fixed at separate positions along the arc of the rotary piece 320 so as to prevent the connecting cables from interfering with each other. More preferably, the rotary piece 320 has a plurality of guide grooves H corresponding to the connecting cables 420 and 620 of the elastic part 400 and the second elastic part 600, respectively, so that the connecting cables 420 and 620 of the elastic part 400 and the second elastic part 600 are individually seated in the guide grooves to be moved along the guide grooves.

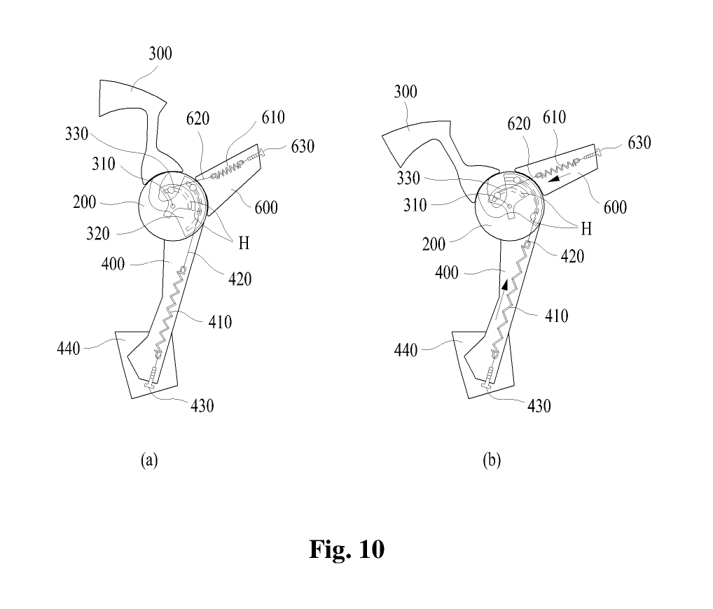

In addition, as illustrated in FIG. 10, the rotating part 300 further includes an auxiliary rotary piece 330 that has the same shape as the rotary piece 320 and is coupled to the rotary shaft 310 to be rotated in a state where it is superposed on the rotary piece 320. Thus, the connecting cable 420 of the elastic part 400 is fixed to the rotary piece 320, and the connecting cable 620 of the second elastic part 600 is fixedly coupled to the auxiliary rotary piece 330, so that the connecting cables 420 and 620 of the elastic part 400 and the second elastic part 600 may be individually connected to the rotating part 300 to be moved.

Thus, the connecting cables 420 and 620 of the elastic part 400 and the second elastic part 600 are operated as the rotating part 300 rotates. The connecting cable 420 of the elastic part 400 moves along the arc of the rotary piece 320 as the rotary piece 320 rotates upwards or downwards, while the connecting cable 620 of the second elastic part 600 moves along the arc of the auxiliary rotary piece 330 as the auxiliary rotary piece 330 rotates upwards or downwards.

The auxiliary rotary piece 330 has a fan shape, and a guide groove H is formed along the arc of the auxiliary rotary piece. Thus, when the rotating part 300 rotates in a state where the connecting cable 620 of the second elastic part 600 is seated in the guide groove H, the auxiliary rotary piece may guide the movement of the connecting cable 620 of the second elastic part 600.

A joint assisting device 1 according to a further embodiment of the present invention includes the wearing part 100, the connecting part 200, the rotating part 300, and the elastic part 400, as in the preceding embodiment.

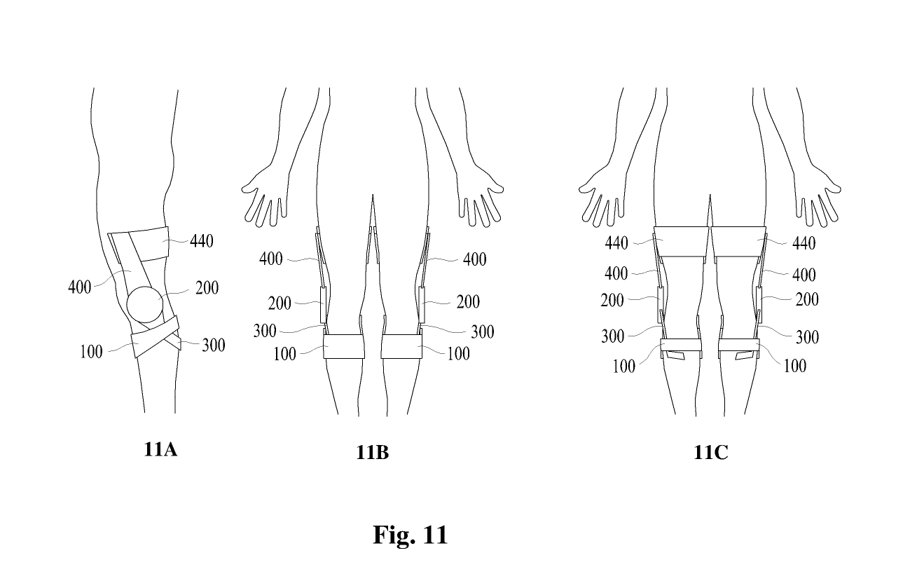

However, as illustrated in FIGS. 11A to 11C, the wearing part 100 comprises a pair of wearing parts to be worn on both legs of a wearer's body. The connecting part 200 is provided on a left or right side of an upper part of each wearing part 100 to be fixed to the knee joint of each leg of the wearer.

The rotating part 300 is worn on the body of a lower side of the wearing part 100 to rotate integrally with the body when the knee is bent or stretched. A rotary shaft 310 is provided on a side of the rotating part to be coupled with the connecting part 200, and the rotating part includes a rotary piece 320 that has a fan shape and rotates around the rotary shaft 310, and a guide groove H is formed along an arc of the rotary piece 320. The details of the respective components may be easily derived from the above-described embodiments.

The elastic part 400 includes an elastic member 410 that is coupled at a lower part thereof to the connecting part 200 to reduce a weight applied to the knee joint when the rotating part 300 rotates and is embedded such that an upper or lower end thereof is fixed, a connecting cable 420 that connects the elastic member 410 with the rotary piece 320 and is moved while being seated in the guide groove H when the rotating part 300 rotates, and an elastic fixing band 440 that is provided on an upper part of the elastic part to be worn on the thigh. The details of the respective components may be easily derived from the above-described embodiments.

As such, the other side of the rotating part 300 is worn on a wearer's body at a lower position of the wearing part 100 so that the rotating part is rotated when a wearer bends or stretches his or her knee. The elastic fixing band 440 is worn on a rear of a wearer's thigh, so that, in a state where the upper part of the elastic part 400 is fixed to the rear of a wearer's thigh, it is operated by the rotation of the rotating part 300, thus supplementing energy consumed in a wearer's knee joint movement through the expansion or compression of the elastic member 410 caused by the rotation of the rotating part 300 when the wearer bends or stretches his or her knee, thereby reducing a weight applied to the joint or allowing a wearer's leg to be more efficiently exercised.

Consequently, the joint assisting device according to the present invention has the relatively simple structure, thus guaranteeing the easy manufacture as well as the convenient handling and use, and reducing the manufacturing cost, and supplements the energy consumed in the lumbar or joints movements through the expansion and compression of the elastic member in a state where force is not directly applied to the joints, thus reducing a weight applied to the joints, and does not require separate manipulation, thus enabling convenient use, and adjusts an angle at which the left and right sides are connected via the fastening member when the device is worn, thus being fit for various body sizes of wearers and applicable to individual motions of left and right sides of the body, and adjusts the tension of the elastic member via the tension adjusting member, thus being efficiently used according to a wearer's height or weight.

While the invention has been shown and described with reference to exemplary embodiments thereof, it will be understood by those skilled in the art that various changes in form and details may be made therein without departing from the spirit and scope of the invention as defined by the appended claims. Therefore, it is to be understood that the scope of the invention is not limited to the above-described embodiments, and all changes that fall within meets and bounds of the claims, or equivalence of such meets and bounds are intended to be embraced by the claims.

As described above, the present invention provides a joint assisting device, which has a relatively simple structure, thus guaranteeing easy manufacture as well as convenient handling and use, and which supplements energy consumed in the lumbar or joints movements by expanding or compressing an elastic member in a state where force is not directly applied to the joints, thus reducing a weight applied to the joints, and which does not require separate manipulation, thus enabling convenient use. As a result, the joint assisting device is industrially available.

1. A joint-assisting device comprising:
a wearing part worn on the waist of a body;
a connecting part provided at each of both sides of a lower part of the wearing part to be fixed around the hip joint;
a rotating part worn on the body at an upper side of the wearing part to rotate while moving integrally with the body during bending or stretching motions of the upper body or knees, coupled to the connecting part by a rotary shaft provided at one side thereof, having a rotary piece formed in a fan shape to rotate around the rotary shaft, and having a guide groove formed along an arc of the rotary piece; and
an elastic part coupled at an upper part thereof to the connecting part to reduce a weight applied to the lumbar or joints when the rotating part rotates, and comprising an elastic member embedded in a state in which an upper or lower end thereof is fixed, a connecting cable connecting the elastic member and the rotary piece and moving in a state in which the connecting cable is seated in the guide groove, when the rotating part rotates, and an elastic fixing band provided at a lower part of the elastic part to be worn on the thigh.

2. The joint-assisting device of claim 1, further comprising:
a second elastic part having an elastic member embedded to be coupled at the other side to the rotary piece, fixed to a lower side of the wearing part, and reducing a weight applied to the lumbar or joints when the rotating part rotates

3. The joint-assisting device of claim 1, wherein each of the wearing part and the rotating part comprises a fastening member connecting both sides thereof,
the fastening member is rotatably provided in a center of each of the wearing part and the rotating part to adjust an angle at which the both sides of each of the wearing part and the rotating part are connected.

4. The joint-assisting device of claim 1, wherein the rotating part is worn on the upper side of the wearing part to extend through the flank to the abdominal region or the back.

5. The joint-assisting device of claim 1, further comprising:
a shoulder wearing part coupled at one side to the wearing part and holding at the other side on the shoulder,
the shoulder wearing part comprises a fastening member that is rotatably provided in a center to connect both sides thereof, thus adjusting an angle at which the both sides are connected, and the rotating part extends at the other side thereof through the flank along the chest or the back to the shoulder to be coupled to the other side of the shoulder wearing part.

6. The joint-assisting device of claim 1, wherein the elastic part further comprises:
a tension adjusting member coupled to an upper or lower end of the elastic member, and pushing the upper end of the elastic member or pulling the lower end of the elastic member, thus adjusting tension of the elastic member.

7. The joint-assisting device of claim 2, wherein an upper part of the elastic part and the other side of the second elastic part are connected to the connecting part, respectively, each elastic part comprising:
an elastic member comprising a tension spring or a compression spring, fixed at an upper end or a lower end thereof to an inside of the elastic part, and embedded in each of the elastic part and the second elastic part in a state where one end or the other end is fixed to an inside of the second elastic part;
a connecting cable fixed to the upper end of the elastic member of the elastic part and the rotary piece, and fixed to the other end of the elastic member of the second elastic part and the rotary piece to connect the rotary piece to the elastic member of each of the elastic part and the second elastic part, and moving along an arc of the rotary piece when the rotating part rotates; and
a tension adjusting member connected to the upper or lower end of the elastic member of the elastic part to push the upper end or pull the lower end of the elastic member of the elastic part, and connected to one end or the other end of the elastic member of the second elastic part to pull one end or push the other end of the elastic member of the second elastic part, thus adjusting tension of the elastic member of each of the elastic part and the second elastic part.

8. The joint-assisting device of claim 7, wherein the connecting cables of the elastic part and the second elastic part are fixed, respectively, to the rotary piece to be spaced apart from each other along the arc of the rotary piece.

9. The joint-assisting device of claim 7, wherein the rotating part further comprises an auxiliary rotary piece having the same shape as the rotary piece and coupled to the rotary shaft to be superposed on the rotary piece,
the connecting cable of the elastic part is fixed to the rotary piece, and
the connecting cable of the second elastic part is fixed to the auxiliary rotary piece.

10. A joint-assisting device comprising:
a pair of wearing parts worn on the legs of a body;
a connecting part provided at a left or right side of an upper part of each of the wearing part to be fixed around the knee joint;
a rotating part worn on the body at a lower side of the wearing part to rotate while moving integrally with the body during bending or stretching motions of the knees, coupled to the connecting part by a rotary shaft provided at one side thereof, having a rotary piece formed in a fan shape to rotate around the rotary shaft, and having a guide groove formed along an arc of the rotary piece; and
an elastic part coupled at a lower part thereof to the connecting part to reduce a weight applied to the knee joint when the rotating part rotates, and comprising an elastic member embedded in a state in which an upper or lower end thereof is fixed, a connecting cable connecting the elastic member and the rotary piece and moving in a state in which the connecting cable is seated in the guide groove, when the rotating part rotates, and an elastic fixing band provided at an upper part of the elastic part to be worn on the thigh.
